(19) United States
(12) Patent Application Publication
(10) Pub. No.: US 2015/0126981 A1
(43) Pub. Date: May 7, 2015

(54) HAIR TREATMENT DEVICE WITH LIGHT-BASED HAIR DETECTOR

(71) Applicant: KONINKLIJKE PHILIPS N.V., EINDHOVEN (NL)

(72) Inventors: Babu Verghese, Eindhoven (NL); Rieko Verhagen, Eindhoven (NL)

(73) Assignee: KONINKLIJKE PHILIPS N.V.

(21) Appl. No.: 14/396,487

(22) PCT Filed: Apr. 12, 2013
(86) PCT No.: PCT/IB2013/052926

§ 371 Date: Oct. 23, 2014

Related U.S. Application Data
(60) Provisional application No. 61/637,354, filed on Apr. 24, 2012.

(51) Int. Cl. A61B 18/20

(52) U.S. Cl. CPC A61B 18/203; A61B 2018/00476
USPC 606/9

(57) ABSTRACT

The invention provides a hair treatment device (40) comprising a detector (10) for detecting a hair (22) near a skin surface (21). The detector (10) comprises a light source (11) for generating a light beam (31) with an initial polarization direction, a polarization modulator (32) for time-dependent modulation of the polarization direction of the light beam (31) between at least a first polarization direction and a second polarization direction, optical elements (14, 16, 17, 18) for focusing the light beam (31) at the hair (22) near the skin surface (21), and a polarization-sensitive light-based detection unit (13) for detecting light interacted with the hair (22) or the skin surface (21) and for discerning when the interacted light has a polarization-direction component different from the initial polarization direction.

### FIELD OF THE INVENTION

This invention relates to a hair treatment device comprising a detector for detecting a hair near a skin surface, the detector comprising a light source for generating a polarized light beam having an initial polarization direction, optical elements for focusing the light beam at the hair near the skin surface, and a polarization-sensitive light-based detection unit for detecting light interacted with the hair or the skin surface and having a polarization-direction component different from the initial polarization direction.

### BACKGROUND OF THE INVENTION

Such a hair treatment device is, e.g., known from the international patent application published as WO 2010/106480 A1. This patent application describes a device for imaging a hair near a skin surface of a body part. The device comprises a light source for generating a light beam with an incident polarization direction, and a sensor for detecting light returning from said hair. The sensor has separate photodiodes for detecting light with the incident polarization (parallel polarized) and light with a polarization direction orthogonal to the incident polarization (cross polarized). The ratio of light intensities detected by the separate photodiodes is a probability measure for the presence of a hair at the tested skin location.

It has turned out that the sensitivity and specificity of this known detector is largely dependent on the angle between the polarization direction of the incident light and the orientation of the hair to be detected and on the focusing depth inside the hair. Because the orientation of the hairs and the focusing depth inside the hair differ from hair to hair and over time, the hair-skin contrast obtained by the known detector is not entirely satisfactory. Furthermore, the orientation of the hair in a plane perpendicular to the polarization direction of the incident light is a critical parameter that determines the sensitivity and specificity of hair detection.

Attempts have been made to reduce the orientation dependency of hair detection systems, e.g. by using elliptically or circularly polarized incident light (WO 2008/072151 A2) or by using radial polarization or other types of time-invariant and spatially variant polarization (not yet published patent application of this applicant, attorney docket no. 2011PF00990). Although these solutions provide clearly improved results, some orientation dependence is still present. For example, the use of radial polarization averages out the orientation dependent variations in the hair-skin contrast instead of making the system completely orientation independent.

### OBJECT OF THE INVENTION

It is an object of the invention to further improve hair-skin contrast for different orientations of the hair.

### SUMMARY OF THE INVENTION

According to a first aspect of the invention, this object is achieved by providing a hair treatment device comprising a detector for detecting a hair near a skin surface, the detector comprising a light source for generating a polarized light beam having an initial polarization direction, a polarization modulator for time-dependent modulation of the polarization direction of the light beam between at least a first polarization direction and a second polarization direction, optical elements for focusing the light beam at the hair near the skin surface, and a polarization-sensitive light-based detection unit for detecting light interacted with the hair or the skin surface and for discerning when the interacted light has a polarization-direction component different from the initial polarization direction.

The birefringent effect of the hair depends on the direction of the incident polarization. When the incident light beam hits a hair at the right angle with respect to the hair, the birefringent properties of the hair cause the polarization of the light traversing through the hair to change. This change of polarization is at a maximum when the polarization direction of the light beam is at a 45° angle with the optical axis of the hair and at a minimum when at a 0° or 90° angle. When the birefringent effect is at a minimum, the polarization dependent light-based detection unit will not discern much light with a polarization-direction component different from the initial polarization direction. When the birefringent effect is at a maximum, the light coming back from the hair and having a polarization-direction component different from the initial polarization will also be at a maximum. By modulating the polarization direction of the light, the different polarization-direction component of the returning light will vary accordingly and will provide the detection signal with a time-dependent component in, e.g., the cross polarized channel.

By detecting this time-dependent component in the returning light, the presence of a hair can be detected. As will be elucidated below, instead of directly detecting, e.g., the cross-polarized component of the interacted light, the presence of a cross-polarized component can also be discerned in other ways. The current invention shows a number of ways of reliably detecting the variations in the different polarization-direction component of the returning light in order to detect the presence of a hair.

In the following, most embodiments will use a periodic modulation of the incident polarization with a certain modulation frequency and the different polarization-direction component to be considered is orthogonal to the initial polarization direction. It is, however, to be noted that the modulation does not have to be periodic and that similar results may be obtained by observing other polarization-direction components of the interacted light. For the most sensitive and accurate detection, periodic modulation and detection of the orthogonal polarization-direction component is preferred.

In a first embodiment of the device according to the invention, the polarization-sensitive light-based detection unit comprises a polarization-sensitive light sensor for selectively detecting the polarization-direction component, different from the initial polarization direction, of the light interacted with the hair or the skin surface. If the polarization direction is modulated periodically, a phase sensitive detection unit may be coupled to the polarization modulator and to the polarization-sensitive light sensor for detecting a phase difference between a sensor signal from the polarization-sensitive light sensor and a reference signal from the polarization modulator, the reference signal having the modulation frequency.

The polarization sensitive light sensor detects, e.g., the cross polarized component of the light returning from the skin or hair. For light returning from a hair, the detected signal shows a maximum at each moment that the incident polarization direction and the hair enclose an angle of 45°. By detecting this AC component of the returning light, the presence of a hair can be detected. When the direction of the incident polarization is modulated at a frequency f, the polarization-sensitive light sensor will generate a periodical detection signal of the same frequency. The detection signal may additionally comprise 2f-components and higher harmonics.

The amplitude of the AC component of the detection signal is at a maximum when the direction of the incident polarization is at an angle of 45° with the optical axis of a hair. Phase sensitive detection of the detection signal makes it possible to accurately detect the AC component, even when the detection signal is very noisy and the AC component is relatively small. The reference signal from the polarization modulator is used by the phase sensitive detection unit for singling out those components of the detection signal having the same frequency as the reference signal. The frequency of the reference signal may be doubled (or tripled, . . . ) for enabling detection of 2f-components and higher harmonics.

In another embodiment, the light source comprises a laser diode, the optical elements being arranged to optically feedback the light interacted with the hair or the skin surface to the laser diode. The polarization-sensitive light based detection unit comprises an oscillation parameter monitoring unit for monitoring an oscillation parameter of the laser diode and for analyzing the oscillation parameter for discerning when the interacted light has the polarization-direction component orthogonal to or otherwise different from the initial polarization direction.

Optical feedback of a light beam into the laser diode changes the oscillation behavior of the laser diode. Many parameters of the oscillation behavior depend on the polarization direction of the feedback light. The positive optical feedback of light to the laser diode and the resulting change of oscillation characteristics influence oscillation parameters, such as e.g. drive current, emission spectrum, threshold voltage, line width, amplitude or onset of instabilities, of the laser diode. When the returning light comes from a hair that is hit at the right angle (45° angle between the optical axis of the hair and the polarization direction of the incident beam) the emitted and returning light have different polarization directions due to the birefringence of the hair, which leads to different oscillation characteristics. When the light beam hits a hair at a ‘wrong’ angle (not 45 , closer to 0° or 90°), the polarization of the returning beam will be closer to that of the emitted one. By analyzing at least one of the relevant oscillation parameters, it can be observed when a hair is detected. When the angle between the polarization direction of the incident light and the optical axis of the hair is modulated with a modulation frequency, the oscillation parameters of the laser diode vary accordingly. The variation in these parameters can be detected in a number of different ways.

In an embodiment, a polarization direction of the light beam is the oscillation parameter and the oscillation parameter monitoring unit comprises a polarization-sensitive light sensor for selectively detecting the polarization-direction component, different from the initial polarization direction, of light emitted by the laser diode.

One of the oscillation parameters that is influenced by the polarization direction of the feedback light is the polarization direction of the emitted light. When the returning light comes from the skin surface, the light emitted by and returning to the laser diode have the same polarization direction. When the returning light comes from a hair that is hit at the right angle (45° angle between the optical axis of the hair and the polarization direction of the incident beam) the emitted and returning light have different polarization directions due to the birefringence of the hair. When the optical feedback returns light with the changed polarization direction to the laser diode, the oscillation behavior of the diode will change and, e.g., the orthogonal component of the polarization direction of the emitted light will increase. This effect is detected by the polarization-sensitive light sensor, which will provide an AC signal when the incident polarization is modulated. When the light beam hits a hair at a ‘wrong’ angle (not 45 , closer to 0° or 90°), the polarization direction of the returning beam will be closer to that of the emitted one and the signal from the polarization-sensitive light sensor decreases or diminishes. When using periodic modulation of the incident polarization direction, variation in the light sensor signal will result in an AC component that is detected using phase sensitive detection, with the reference signal from the polarization modulator as a reference.

The oscillation parameter monitoring unit may comprise a phase sensitive detection unit, coupled to the polarization modulator and arranged for detecting a phase difference between the oscillation parameter and a reference signal from the polarization modulator, the reference signal having the modulation frequency. With such a phase sensitive detector, small variations of the oscillation parameter can reliably be detected.

These and other aspects of the invention are apparent from and will be elucidated with reference to the embodiments described hereinafter.

### BRIEF DESCRIPTION OF THE DRAWINGS

In the drawings:

FIG. 1 schematically shows an exemplary hair detector according to the invention,

FIG. 2 schematically shows a hair detector with an optical feedback loop,

FIG. 3 schematically shows a different hair detector with optical feedback, and

FIG. 4 schematically shows a hair treatment device with a hair detector.

### DETAILED DESCRIPTION OF THE INVENTION

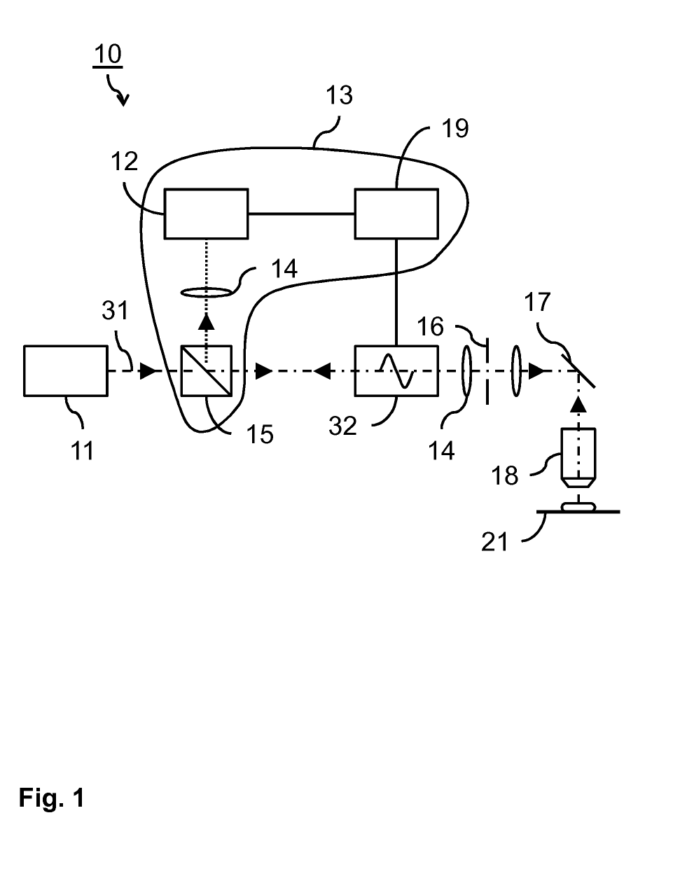

FIG. 1 schematically shows an exemplary hair detector 10 according to the invention. In principle the detector 10 is suitable for detecting any kind of birefringent object at any surface. For example, the detector 10 may be adapted to detect collagen in human or animal skin 21 for determining skin elasticity. However, in the following, the detector 10 will be used for detecting hairs on a human or animal skin surface 21. Hair detection may be useful in, IPL (Intense Pulsed Light) based or laser based shaving apparatuses. The hair detector 10 of FIG. 1 comprises a laser source 11 for emitting a laser beam 31, preferably in the near-infrared or infrared part of the spectrum. Typically, light with a wavelength between 400 and 2000 nm is used. Wavelengths in the range 600-1064 nm are preferred. For example, an Nd:YAG laser diode may be used. Optical elements, like lenses 14, pin holes 16 and/or mirrors 17 lead the beam 31 to an objective for focusing the light beam 31 at or near the skin 21. A control unit (not shown) coupled to the laser source 11 and/or (part of) the optical elements 14, 17 controls the exact optical path of the laser beam 31 in order to control the exact area of skin 21 that is tested for the presence of a hair 22 and to enable scanning lines or 2D areas of skin 21.

The light beam 31 reaching the skin 21 will be partly returned through scattering, reflection and other interactions. The returning light enters the detector 10 through the same objective 18 and is analyzed for finding out whether there is a hair at the focus or not. For this analysis, the birefringent effect of hair is used. When the incident light beam 31 hits a hair at the right angle with respect to the hair, the birefringent properties of the hair cause the polarization of the light traversing through the hair to change. The light source 11 of the hair detector 10 according to the invention provides light 31 with a certain initial polarization direction in order to make it possible to detect this change of polarization. A polarization-sensitive light-based detection unit 13 may be used for detecting this change of polarization and determining when a hair is present at the focus position.

The change of polarization is at a maximum when the polarization of the light beam 31 is at a 45° angle with the hair and at a minimum when at a 0° or 90° angle. The orientation of the hair relative to the skin 21 and the detector 10 is, however, different for different hairs and may change while the hair treatment device is moved along the skin surface 21. The hair detector 10 of FIG. 1 therefore comprises a polarization modulator 32 for modulating the polarization of the light beam between a first and a second polarization direction. The modulation is preferably of a periodic nature and with a fixed modulation frequency. However, also other variations of the incident polarization may be used. The polarization modulator 32 may, e.g., be a piezo-elastic or electro-optic modulator, capable of modulating the polarization of the light beam at high speeds. In a piezo-elastic modulator the polarization components parallel or perpendicular to the modulator axis travel at slightly different speeds as the piezo-elastic material is modulated. The phase difference thus created between the two components oscillates, e.g., sinusoidally as a function of time. The angle between the first and the second polarization direction is preferably at least 45°, such that for most hairs there will be at least one configuration in which the angle between the polarization direction and the optical axis of the hair is 45°. With a modulating range of about 90°, such a configuration is provided for all hairs and in all possible orientations.

The detector 10 of FIG. 1 comprises a polarization-sensitive light-based detection unit 13 for selectively detecting a different, preferably orthogonally, polarized component of the returning light. Orthogonally polarized therein means with a polarization orthogonal to the initial polarization direction of the light provided by the light source 11. In this embodiment the detection unit 13 comprises a polarizing beam splitter 15, a light sensor 12 and a phase sensitive detector 19. The light returning from the skin 21 or hair is directed to the light sensor 12 by optical elements, such as mirrors 17, lenses 14 and a polarizing beam splitter 15. Due to the polarizing beam splitter 15 and/or a polarization filter (not shown), the light sensitive sensor 12 only detects light with a selected polarization component different from the initial polarization. When the birefringent effect is at a minimum (0° or 90° angle between polarization direction and optical axis of the hair) or when the light returns from the skin surface instead of from a hair, the differently polarized component in the returning light is small or even non-existent. When the birefringent effect is at a maximum (45° angle between polarization direction and optical axis of the hair), the differently polarized component in the returning light is also at a maximum. When the polarization of the incident light beam 31 is modulated at a modulation frequency f, the light sensor 12 will measure this maximum signal periodically at the same frequency f When modulating the polarization direction of the light, the orthogonal component of the returning light will thus vary accordingly and the detection signal will comprise an AC component in the cross polarized channel. By detecting this AC component in the returning light, the presence of a hair can be detected.

In principle, many different signal analyzing methods can be used for determining when the sensor signal indicates that a hair is found. For example, a simple circuit for checking whether the orthogonal component reaches a certain threshold may already be sufficient. In this embodiment, a phase sensitive detector 19 is used. The phase sensitive detector 19 uses a reference signal from the polarization modulator 32. The reference signal has the same frequency as the frequency of the polarization modulation. The phase sensitive detector 19 determines a phase difference between the reference signal and the sensor signal. At those locations where the phase difference is found to be maximal as the incident polarization is temporarily varied, a hair is present. A lock-in amplifier may be used for the phase sensitive detection.

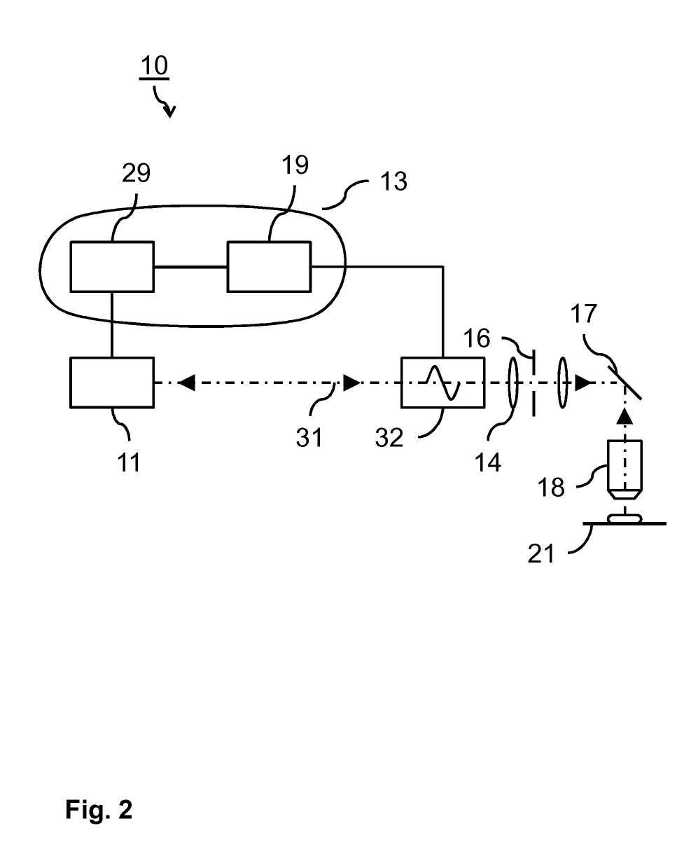

FIG. 2 schematically shows a hair detector 10 with an optical feedback loop. In this embodiment, the change in polarization induced by the presence of hair is optically feedback into the laser 11 and this will result in a change in the oscillation behavior and emission spectrum of the laser 11. Many of the oscillation characteristics of the laser source such as output polarization, threshold current, emission spectrum and terminal voltage will depend on the optical feedback. These output fluctuations can be monitored for detecting the presence of the hair based on the depolarization effects induced by the presence of hair. In this embodiment, the drive current and/or other operational parameters are monitored by laser driver monitoring means 29. Like in FIG. 1, a phase-sensitive detector 19, using the reference signal from the polarization modulator 32, may be used for detecting a phase difference between the fluctuating drive current signal and the reference signal in order to determine when a hair is detected.

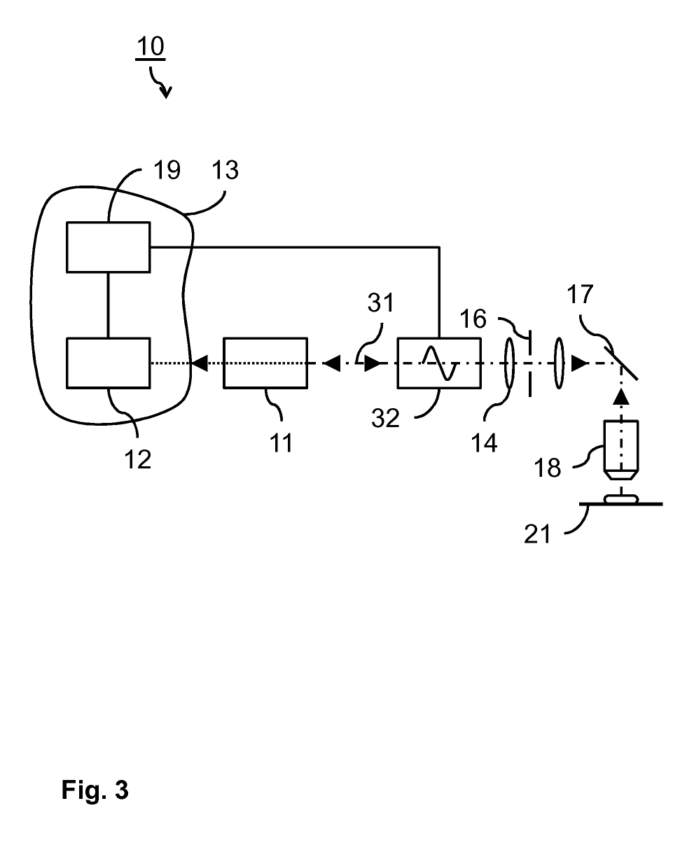

Also the embodiment of FIG. 3 uses optical feedback for detecting the AC component in the cross polarized channel. In this embodiment, the change in polarization induced by the presence of hair is optically feedback into the laser 11 and this will result in a polarization change in the emission spectrum of the laser 11. In this exemplary embodiment, the rear face of the laser diode is antireflection coated. The reflected/scattered light detected from the hair is optically coupled to the laser diode. The laser power from the uncoated front face is used to record the oscillation behavior using a polarization sensitive light detector 12 comprising, e.g., a polarization analyzer and a photodiode. When the returning light is reflected/scattered from skin, the optical feedback provides light with the incident polarization and the polarization sensitive light detector 12 detects a more or less stable DC signal. When the laser beam 31 is focused at a hair, the polarization modulator will cause the birefringent effect of the hair to vary with the polarization of the incident beam. When the polarization of the incident beam 31 is at the right angle (45°) with the optical axis of the hair, the returning light beam will have an increased orthogonal component and a decreased parallel component. As a result, the oscillation characteristics of the laser diode change. The laser diode emits more light with the orthogonal polarization and the signal from the light detector 12 increases. The periodic variation in the detector 12 signal indicates the presence of a hair. Like in the previous figures, a phase sensitive detector 19 coupled to the polarization modulator 32, may be used for reliably detecting the periodic variations with the same frequency as, or an integer multiple of, the modulation frequency of the polarization modulator 32.

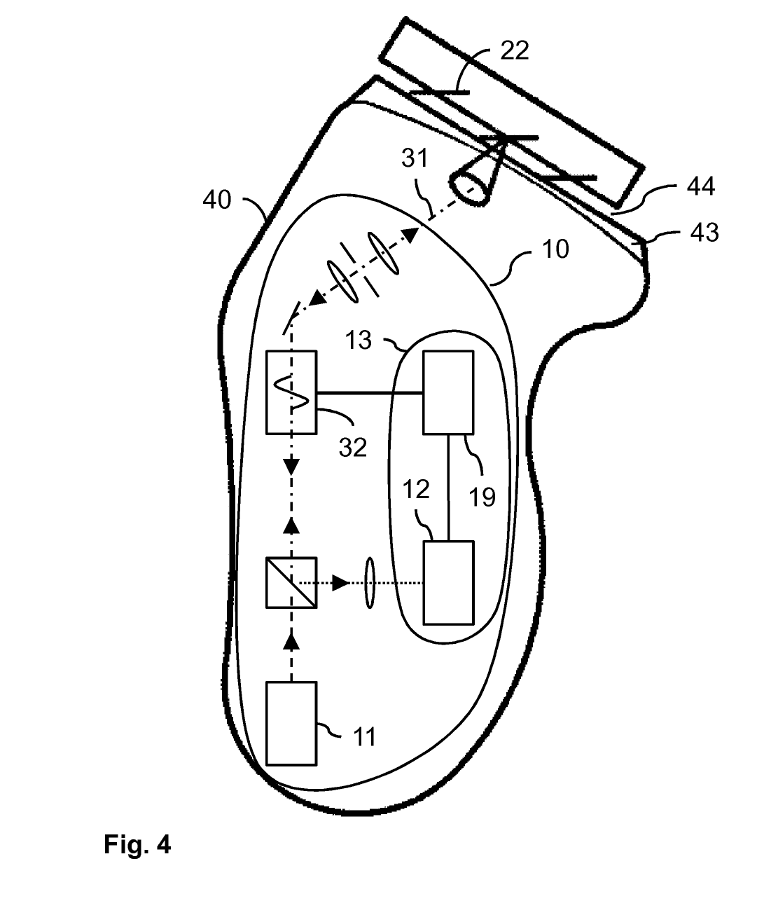

FIG. 4 schematically shows a shaving device 40 with a hair detector 10. The shaving device 40 comprises a hair detector 10 similar to the one described above with reference to FIG. 1. Equal reference numbers correspond to similar features. In addition to features already discussed above, the shaving device 40 may also comprise an optical or contact window 43 and an immersion fluid 44 for improving the penetration properties of the radiation into the skin 21. For example, the fluid 84 may be an index matching fluid, having an index of refraction which is halfway between that of the optical window and that of the skin 21. Preferably, all refractive indices are substantially equal. The fluid 44 may also be selected for the purpose of cooling the skin 21, or treating it otherwise. Furthermore, although the contact window 43 is optional, it helps in serving as a reference for determining positions of skin objects, such as the hairs 22.

The shaving device 40 may not only use the laser source 11 for detecting the hair 22, but also for cutting it. When the laser source 11 is used for cutting, it may operate at a different power level than when detecting hairs 22. Alternatively, a separate laser source (not shown) is used for the cutting of the hairs 22. The control over the cutting process may be performed by the control unit or by an additional cutting processor (not shown). The cutting processor is coupled to the light based detector 10 to activate the hair-cutting laser source in a focal position of the hair-cutting laser beam near the skin surface 21 in which the light-based detector has detected the presence of a hair 22.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A hair treatment device comprising a detector for detecting a hair near a skin surface, the detector comprising:
a light source for generating a polarized light beam having an initial polarization direction,
a polarization modulator for time-dependent modulation of the polarization direction of the light beam between at least a first polarization direction and a second polarization direction,
optical elements configured and arranged to lead the light beam from the polarization modulator to an objective for focusing the light beam at the hair near the skin surface, the optical elements being further configured and arranged to return light interacted with the hair or the skin surface to the polarization modulator through the objective; and
a polarization-sensitive light-based detection unit configured and arranged for detecting the light, interacted with the hair or the skin surface and returned through the objective and through the polarization modulator, and for discerning when the interacted light has a polarization-direction component different from the initial polarization direction.

2. A hair treatment device as claimed in claim 1, wherein the polarization-direction component different from the initial polarization direction is orthogonal to the initial polarization direction.

3. A hair treatment device as claimed in claim 1, wherein the time-dependent modulation is a periodic modulation with a modulation frequency.

4. A hair treatment device as claimed in claim 3, wherein the polarization-sensitive light-based detection unit comprises:
a polarization-sensitive light sensor for selectively detecting the polarization-direction component, different from the initial polarization direction, of the light interacted with the hair or the skin surface, and
a phase sensitive detection unit, coupled to the polarization modulator and to the polarization-sensitive light sensor, for detecting a phase difference between a sensor signal from the polarization-sensitive light sensor and a reference signal from the polarization modulator, the reference signal having the modulation frequency.

5. A hair treatment device as claimed in claim 1, wherein
the light source comprises a laser diode, the optical elements being arranged to optically feedback the light interacted with the hair or the skin surface to the laser diode, and wherein
the polarization-sensitive light-based detection unit comprises an oscillation parameter monitoring unit for monitoring an oscillation parameter of the laser diode and for analyzing the oscillation parameter for discerning when the interacted light has the polarization-direction component different from the initial polarization direction.

6. A hair treatment device as claimed in claim 5, wherein a drive current of the laser diode is the oscillation parameter and wherein the oscillation parameter monitoring unit is arranged to monitor the drive current.

7. A hair treatment device as claimed in claim 5, wherein an emission spectrum of the laser diode is the oscillation parameter and wherein the oscillation parameter monitoring unit is arranged to monitor the emission spectrum.

8. A hair treatment device as claimed in claim 5, wherein a threshold voltage of the laser diode is the oscillation parameter and wherein the oscillation parameter monitoring unit is arranged to monitor the threshold voltage.

9. A hair treatment device as claimed in claim 5, wherein a polarization direction of the light beam is the oscillation parameter and wherein the oscillation parameter monitoring unit comprises a polarization-sensitive light sensor for selectively detecting the polarization-direction component, different from the initial polarization direction, of light emitted by the laser diode.

10. A hair treatment device as claimed in claim 7, wherein the time-dependent modulation is a periodic modulation with a modulation frequency and the oscillation parameter monitoring unit comprises a phase sensitive detection unit, coupled to the polarization modulator and being arranged to detect a phase difference between the oscillation parameter and a reference signal from the polarization modulator, the reference signal having the modulation frequency.

11. A hair treatment device as claimed in claim 1, wherein the second polarization direction includes an angle of at least 45 degrees with respect to the first polarization direction.

12. A hair treatment device as claimed in claim 1, wherein the polarization modulator is a piezo-elastic or electro-optic modulator.

13. A hair treatment device according to claim 1, further comprising a hair-cutting laser source for generating a hair-cutting laser beam and a processor which is coupled to the light-based detection unit, wherein the processor is arranged to activate the hair-cutting laser source in a focal position of the hair-cutting laser beam near the skin surface in which the light-based detection unit has detected the presence of a hair.
